(19) United States
(12) Patent Application Publication
(10) Pub. No.: US 2013/0226058 A1
(43) Pub. Date: Aug. 29, 2013

(54) MOBILITY DEVICE

(71) Applicants: Michael R. Sanders, Provo, UT (US); Jonathan Mark Roberts, Frisco, TX (US); Benjamin Scott Avery, Bountiful, UT (US)

(72) Inventors: Michael R. Sanders, Provo, UT (US); Jonathan Mark Roberts, Frisco, TX (US); Benjamin Scott Avery, Bountiful, UT (US)

(21) Appl. No.: 13/856,391

(22) Filed: Apr. 3, 2013

Related U.S. Application Data
(63) Continuation-in-part of application No. 13/631,741, filed on Sep. 28, 2012.
(60) Provisional application No. 61/540,938, filed on Sep. 29, 2011.
(60) Provisional application No. 61/617,458, filed on Mar. 29, 2012.
(60) Provisional application No. 61/664,660, filed on Jun. 26, 2012.

(51) Int. Cl. A61F 5/01

(52) U.S. Cl. CPC A61F 5/0102
USPC 602/23

(57) ABSTRACT

Mobility devices are provided that enable a user to walk when the user's lower leg is in a non-functioning condition. A mobility device includes a main body, a lower leg support, an upper leg support, and a foot. The main body can be secured to a lateral side of the user's upper leg with the upper leg support. The lower leg support is connected to the main body and supports the user's lower leg. The foot is connected to a lower end of the main body and is configured to engage the ground. The foot may have a split foot configuration with a first portion and a second portion. The first portion of the foot is positioned rearward of the main body and the second portion of the foot is positioned forward of the main body. Each of the first and second foot portions may have an arcuately shaped profile.

### CROSS-REFERENCE TO RELATED APPLICATIONS

This application is a continuation-in-part of U.S. patent application Ser. No. 13/631,741, filed Sep. 28, 2012, and entitled MOBILITY DEVICE, which claims priority to and the benefit of: U.S. Provisional Patent Application Ser. No. 61/540,938, filed Sep. 29, 2011, and entitled BIO-MIMIC MOBILITY AID; U.S. Provisional Patent Application Ser. No. 61/617,458, filed Mar. 29, 2012, and entitled MOBILITY DEVICE; and U.S. Provisional Patent Application Ser. No. 61/664,660, filed Jun. 26, 2012, and entitled MOBILITY DEVICE. This application also claims priority to and the benefit of International Application No. PCT/US2012/58160, filed Sep. 29, 2012, and entitled MOBILITY DEVICE, which claims priority to and the benefit of: U.S. Provisional Patent Application Ser. No. 61/540,938, filed Sep. 29, 2011, and entitled BIO-MIMIC MOBILITY AID; U.S. Provisional Patent Application Ser. No. 61/617,458, filed Mar. 29, 2012, and entitled MOBILITY DEVICE; and U.S. Provisional Patent Application Ser. No. 61/664,660, filed Jun. 26, 2012, and entitled MOBILITY DEVICE. Each of the foregoing applications is incorporated herein by reference in its entirety.

### BACKGROUND OF THE INVENTION

1. Technical Field

The present disclosure generally relates to mobility aid devices. More specifically, the present disclosure relates to mobility aid devices that may be secured to a user's leg to provide mobility to the user despite the user's lower leg being in a non-functioning condition.

2. The Relevant Technology

In the year 2006 alone, over 4.5 million lower leg, ankle, foot, and toe injuries were treated in emergency rooms in the United States. Often such injuries or other events, such as surgery, render the anatomy below the knee joint at least temporarily inoperable. For instance, the lower leg may not be able to bear weight or the ankle or foot may not be able to bend. As a result, the mobility of such individuals may be limited.

In an effort to provide greater mobility to such individuals, various mobility devices have been developed. Some of the most common mobility devices include wheelchairs, knee scooters, and crutches. Although these devices do provide some mobility to individuals with non-functioning lower legs, the extent of that mobility remains limited.

Many different types of terrain must be traversed since there are a wide variety of activities in which individuals engage themselves. Often the activities in which individuals are involved require them to traverse uneven surfaces or fit in, be seated in, navigate, or otherwise operate in confined spaces. Due to their size and operation requirements, it can be difficult with wheelchairs, knee scooter, or crutches to maneuver through narrow spaces, climb or descend stairs, and the like. Additionally, many activities also require the use of the upper extremities or other parts of the body. Because wheelchairs, knee scooters, and crutches typically require the user's hands to operate, it may be difficult for the user to carry objects, open doors, and the like while using a wheelchair, knee scooter, or crutches. Furthermore, the time available or the speed at which some activities are carried out can be important to successfully carrying them out. Common mobility devices may hinder an individual's ability to perform some activities quickly enough.

Therefore, it would be desirable to provide a mobility device for use with the body which substantially mimics the lower leg's natural motion and ability to traverse various types of terrain, is capable of reasonable operation and storage in confined spaces, allows for the use of the upper extremities during operation, allows for similar speeds as is typical of a functioning lower extremity, and does not substantially limit a user's ability to carry out activities for extended periods of time.

### BRIEF SUMMARY OF THE INVENTION

Mobility aid devices are provided herein that may be secured to a user's leg to provide mobility to the user despite the user's lower leg being in an at least partially non-functioning condition. An individual's lower leg may be in an at least partially non-functioning condition as a result of a broken bone (e.g., tibia, fibula, ankle bones, foot bones), Achilles tendonitis, shin splints, sprains, contusions, blood vessel injuries and diseases, wounds, including surgical wounds, and the like. While such conditions may not completely disable the individual's lower leg, such conditions may limit or prevent the lower leg from bearing weight, the ankle or foot from bending, and the like.

According to one example embodiment, a mobility device includes a main body, a lower leg support, one or more upper leg supports, and a foot. The main body has a lower end, an upper end, and a longitudinal axis extending therebetween. The main body may be selectively securable to a lateral side of a user's leg. The lower leg support is connected to the main body and is configured to have a portion of the user's lower leg positioned thereon and selectively secured thereto. The one or more upper leg supports are also connected to the main body. The one or more upper leg supports are configured to selectively secure the upper end of the main body to a portion of the user's upper leg. The foot is connected to the lower end of the main body and is designed to engage the ground or other surface on which the user may stand or walk.

Various aspects or components of a mobility device may be selectively adjustable. For instance, the lower leg support may be movably connected to the main body such that the distance between the lower leg support and the foot is selectively adjustable. Similarly, at least one of the one or more upper leg supports may be movably connected to the main body so that the height of the upper leg support may be selectively adjusted to accommodate users of different heights or leg lengths.

The foot, whether used in connection with the mobility device or not, may take a variety of forms or may have various features or characteristics, or combinations thereof. In some embodiments, for instance, the foot has a split foot configuration with a first or rearward portion and a second or forward portion. In embodiments where the foot is used in connection with a mobility device, the first or rearward portion of the foot may be positioned rearward of the main body (i.e. towards the posterior). Similarly, the second or forward portion of the foot may be positioned forward of the main body (i.e. towards the anterior).

One or both of the first and second portions of the foot may have a generally arcuately shaped profile. The foot may include a generally planar middle region between the first and second portions and which is vertically spaced apart from a support surface on which the foot rests. The foot may also have a central longitudinal axis that extends between a forward end and a rear end. When the foot is used with a mobility device, the central longitudinal axis of the foot may be laterally offset from a longitudinal axis of the main body of the mobility device. In other embodiments, the foot may include a first foot portion that has a generally C-shaped profile and, optionally, a second foot portion connected to a lower end of the first foot portion.

These and other objects and features of the present invention will become more fully apparent from the following description and appended claims, or may be learned by the practice of the invention as set forth hereinafter.

### BRIEF DESCRIPTION OF THE DRAWINGS

To further clarify the above and other advantages and features of the present invention, a more particular description of the invention will be rendered by reference to specific embodiments thereof which are illustrated in the appended drawings. It is appreciated that these drawings depict only illustrated embodiments of the invention and are therefore not to be considered limiting of its scope. The invention will be described and explained with additional specificity and detail through the use of the accompanying drawings in which:

FIG. 1 illustrates a perspective view of an exemplary embodiment of a mobility device in use;

FIG. 2 illustrates a side view of the mobility device of FIG. 1;

FIG. 3 illustrates a rear perspective view of a lower leg cradle of the mobility device of FIG. 1;

FIG. 4 illustrates a top view of the mobility device of FIG. 1;

FIG. 5 illustrates a bottom view of the mobility device of FIG. 1;

FIG. 6 illustrates a perspective view of another exemplary embodiment of a mobility device;

FIG. 7 illustrates a perspective view of an alternative embodiment of an upper leg support cradles for use with a mobility device;

FIG. 8 illustrates a top view of the upper leg support cradle of FIG. 7;

FIG. 9 illustrates a perspective view of yet another alternative embodiment of an upper leg support for use with a mobility device; and

FIG. 10 illustrates a bottom view of an alternative embodiment of a lower leg support and platform for use with a mobility device.

### DETAILED DESCRIPTION

Mobility devices are provided herein that may be secured to a user's leg to provide mobility to the user despite the user's lower leg being in an at least partially non-functioning condition. Such mobility devices may substantially mimic the function of the user's lower leg when the lower leg is in a functioning condition. For instance, at least one embodiment of the disclosed mobility devices may allow the user to walk and engage in other activities, even in confined spaces and over uneven terrain, in a manner substantially similar to how the user would if his or her lower leg were fully functional. At least one embodiment of the disclosed mobility devices may allow at least one of these features without the use of a user's arm to hold the mobility device.

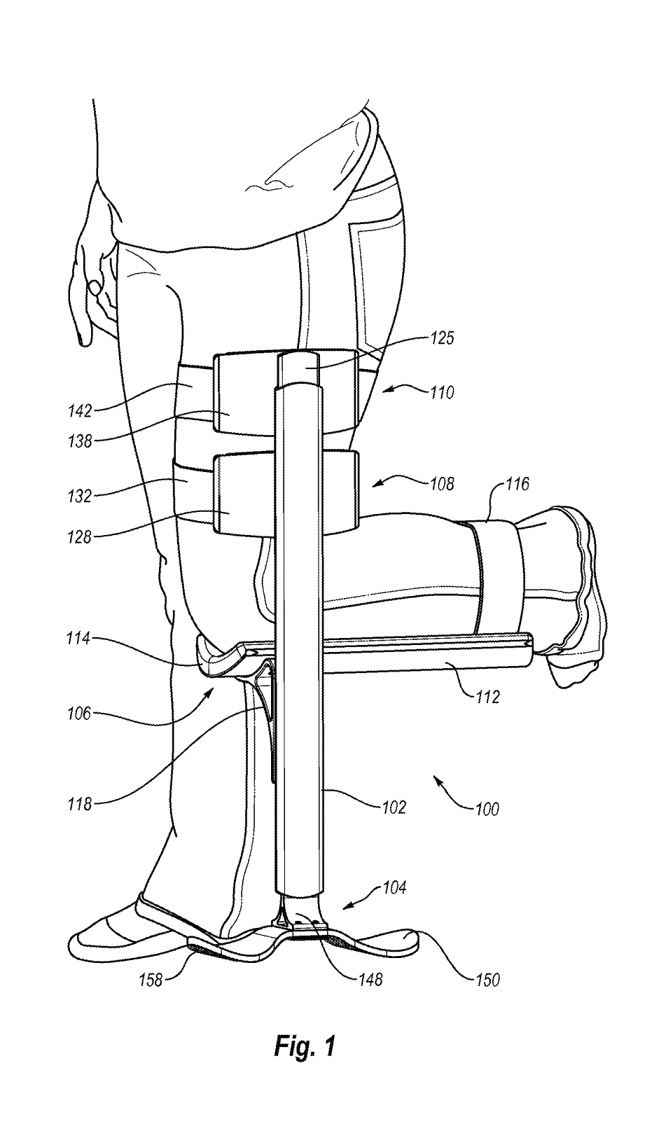

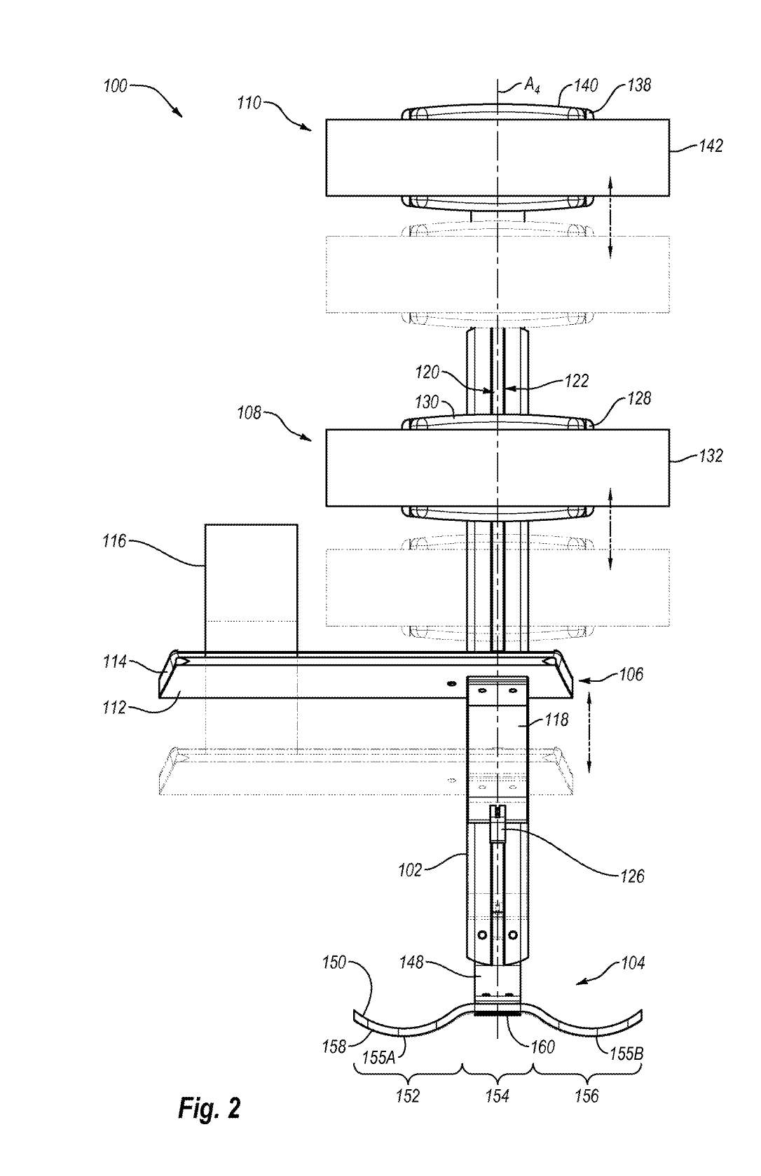

With reference to FIGS. 1 and 2, a mobility device 100 according to one exemplary embodiment is illustrated. In FIG. 1, mobility device 100 is shown in use, while FIG. 2 illustrates mobility device 100 separate from a user. As shown in FIG. 1, mobility device 100 may be secured to a user's leg while the leg is bent at the knee. A portion of mobility device 100 extends from the user's leg to the ground or other support surface. With mobility device 100 secured to the user's leg, mobility device 100 may bear some or all of the user's weight while the user is standing, walking, or engaging in other activities.

As discussed in greater detail below, mobility device 100 includes a main body 102 with a foot 104, a lower leg support 106, a first upper leg support 108, and a second upper leg support 110 connected thereto. Foot 104 is designed to engage the ground or other surface when the user is walking or engaging in other activities while wearing mobility device 100. Lower leg support 106 is designed to have the user's lower leg positioned thereon and secured thereto as shown in FIG. 1. Lower leg support 106 may support the user's lower leg when mobility device 100 is in use. That is, at least some of the user's weight may be transferred from the user's leg to mobility device 100 through lower leg support 106. First upper leg support 108 and second upper leg support 110 are designed to secure mobility device 100 to the user's upper leg and/or waist. Thus the mobility device may transmit these forces from the ground to the user's upper leg through the knee and/or the upper leg support(s) 108, 110. This may reduce the pain experienced by the user due to bearing weight on the user's lower leg.

With regard to lower leg support 106, lower leg support 106 includes a lower leg cradle 112 that is designed to receive or have positioned thereon at least a portion of the user's lower leg and knee. As illustrated, lower leg cradle 112 is curved so that opposing sides thereof are vertically offset from a central region. Having raised sides assists with maintaining the user's lower leg on lower leg support 106 by limiting side-to-side movements of the lower leg relative to lower leg support 106. Additionally, the curved shape of lower leg cradle 112 generally corresponds to the shape of the user's lower leg, which provides greater comfort for the user.

Lower leg support 106 may include cushioning 114 disposed on or in lower leg cradle 112. Cushioning 114 may provide a more comfortable surface for the user's lower leg and knee to rest on. Cushioning 114 or another material on lower leg cradle 112 may also provide a nonslip surface to limit side-to-side and/or fore and aft movements of the lower leg relative to lower leg support 106.

A fastener 116 may be used to secure the user's lower leg to lower leg support 106. In the illustrated embodiment, fastener 116 takes the form of a strap that extends from opposing sides of lower leg cradle 112 and over a portion of the user's lower leg and/or a cast on the user's lower leg. Fastener 116 may be secured around the user's lower leg and/or a cast on the user's lower leg using any of a number of fastening mechanisms and/or combinations thereof, including hook and loop fabrics (e.g., VELCRO), buckles, including slide release buckles, grommets, hooks, zippers, snaps, buttons, and the like.

By way of non-limiting example, fastener 116 may include a strap connected to each side of lower leg cradle 112. Ends of the straps may be connected together around the user's leg with a buckle. The length of one or both of the straps may be selectively adjusted so that fastener 116 may be selectively sized to fit legs of various sizes. The length of one or both of the straps may be adjusted with hook and loop fabrics (e.g., VELCRO). For instance, one of the straps may extend through part of the buckles and be folded back onto itself. The hook and loop fabrics may secure the strap at the desired length. Once the lengths of the straps have been adjusted as desired, the user may simple use the buckles to secure the straps around their leg. As a result, a user may adjust the size of fastener 116 once, and thereafter user the buckle to quickly secure or release the straps around their leg.

Although not illustrated, lower leg support 106 may include more than one fastener 116. For instance, one fastener 116 may extend around the user's calf while a second fastener 116 extends around the user's lower leg near the ankle. As illustrated, the fastener 116 may be located near the user's ankle. In other embodiments, the fastener 116 or fasteners may be located at other positions along the user's lower leg and/or may be selectively movable along the length of lower leg support 116.

Lower leg cradle 112 is mounted on a platform 118. Although lower leg cradle 112 and platform 118 are illustrated as two discrete components that are connected together, it is understood that lower leg cradle 112 and platform 118 may be integrally formed as a single component. Platform 118 is connected to main body 102, thereby linking lower leg cradle 112 with main body 102.

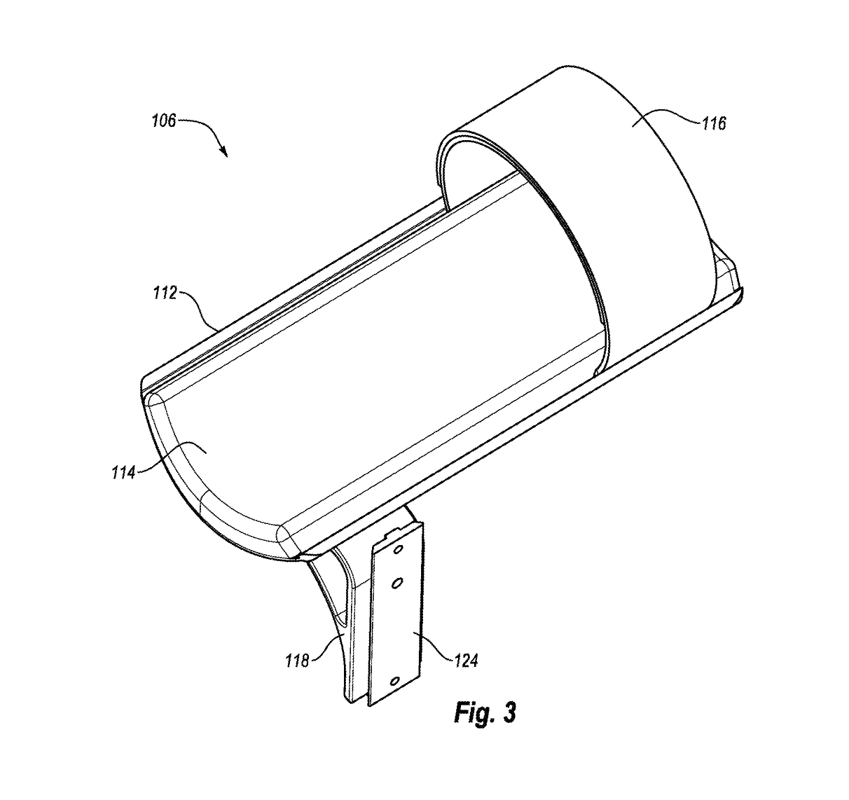

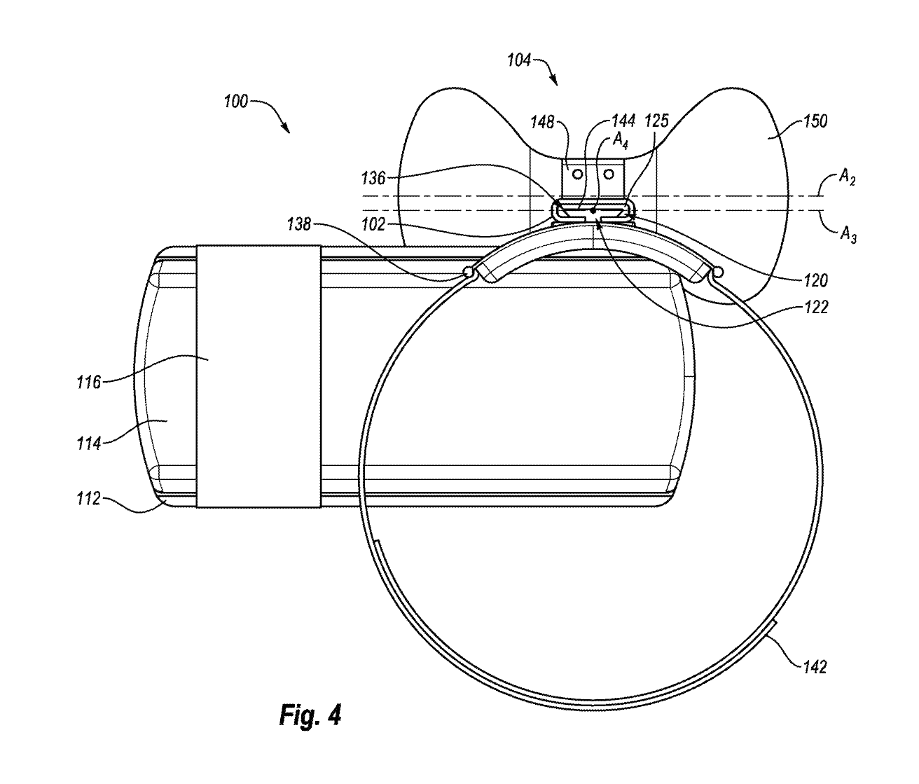

To accommodate users of different heights, the height of lower leg support 106 relative to the ground may be adjusted (i.e. the height from the ground to the base of lower leg support 106). Platform 118 may be movably connected relative to main body 102 to facilitate the height adjustment of lower leg support 106. Platform 118 may be movably connected relative to main body 102 in a variety of ways. For instance, as shown in FIGS. 2 and 4, main body 102 includes a channel 120 therethrough and a slot 122 that extends through a side wall of main body 102 from an outer surface thereof to channel 120. A platform slider 124, as seen in FIG. 3, is slidably positioned within channel 120. Platform 118 is connected to platform slider 124 through slot 122, thereby connecting platform 118 to main body 102.

In order to adjust the height of lower leg support 106 or move lower leg support 106 relative to main body 102, a locking mechanism 126 is loosened or released. With locking mechanism 126 loosened or released, the position of lower leg support 106 relative to main body 102 can be adjusted. By way of example, loosening or releasing locking mechanism 126 may allow platform slider 124 to slide through at least a portion of channel 120. Since lower leg support 106 is connected to platform slider 124, the movements thereof are linked together. Thus, as platform slider 124 moves through channel 120, the position of lower leg support 106 relative to main body 102 is adjusted. Once lower leg support 106 is positioned as desired, locking mechanism 126 can be tightened or reengaged to secure lower leg support 106 in place.

Locking mechanism 126 may take a variety of forms. For instance, locking mechanism 126 may include a quick release bolt. The quick release bolt may be connected between platform 118 and platform slider 124. Tightening the quick release bolt may draw platform 118 and platform slider 124 closer together, with a portion of the main body 102 sidewall positioned therebetween. As the quick release bolt is tightened, the friction between platform 118 and platform slider 124 and the side wall of the main body 102 increases, thereby preventing platform 118 and platform slider 124 from moving relative to main body 102. When the quick release bolt is loosened, the friction between platform 118 and platform slider 124 and the side wall of main body 102 decreases, thereby allowing platform 118 and platform slider 124 to move relative to main body 102. Other example locking mechanisms include screws, bolts, retractable pins and apertures, and the like.

An extension bar 125 is slidably disposed at least partially within channel 120 and includes a track 136 (FIG. 4). Lower leg support 106 may be associated with extension bar 125. For instance, a portion of platform slider 124 may be slidably positioned within track 136 such that extension bar 125 may be slidably repositioned relative to lower leg support 106. Once extension bar is positioned as desired relative to lower leg support 106, lower leg support 106 and extension bar 125 may be selectively secured together with a fastener such as a set screw. When lower leg support 106 and main body 102 are secured together, movement of lower leg support 106 relative to main body 102 also results in movement of extension bar 125 relative to main body 102.

As shown in the Figures, lower leg support 106 may be connected to main body 102 such that lower leg cradle 112 forms a generally right angle with main body 102. It is understood, however, that this is merely exemplary. In other embodiments, lower leg support 106 may be connected to main body 102 such that lower leg cradle 112 forms a an acute or an obtuse angle with main body 102. Furthermore, lower leg support 106 may be connected to main body 102 such that the angle formed between lower leg cradle 112 and main body 102 may be selectively adjusted. This may facilitate users with lower leg injuries that may not allow for the lower leg to be positioned at a right angle to the upper leg.

As noted above, first upper leg support 108 and second upper leg support 110 are designed to secure mobility device 100 to the user's upper leg and/or waist. First upper leg support 108 includes a first upper leg cradle 128 that is designed to be positioned against the user's upper leg. First upper leg cradle 128 may be curved so as to extend at least partially around the user's upper leg. Curving first upper leg cradle 128 may assist with maintaining the desired positioning of the user's upper leg relative to the first upper leg support 108 by limiting fore and aft movements of the upper leg relative to first upper leg support 108.

First upper leg support 108 may include cushioning 130 disposed on or in first upper leg cradle 128. Cushioning 130 may provide a more comfortable surface for the user's upper leg. Cushioning 130 or another material on first upper leg cradle 128 may also provide a nonslip surface to limit fore and aft movements of the user's upper leg relative to first upper leg support 108. Additionally, a fastener 132 may be used to secure the user's upper leg to first upper leg support 108. Fastener 132 may be similar or identical to fastener 116. The first and/or second upper leg cradles 128, 138 may be preformed with a desired curvature. In other embodiments, the first and/or second upper leg cradles 128, 138 may be adjustable to fit a desired curvature to accommodate users with larger and/or smaller upper legs, including users that may have an upper leg in a cast or other upper leg enlarging device.

Similar to lower leg support 106, the height of first upper leg support 108 may be selectively adjustable to accommodate users of different heights and/or leg lengths. By way of example, a first upper leg support slider 134 (not shown, but which may be similar or identical to platform slider 124) may be movably connected to extension bar 125. For instance, first upper leg support slider 134 may be slidably received within track 136. First upper leg support 108 may be repositioned relative to extension bar 125 by sliding first upper leg support slider 134 within track 136. Once first upper leg support 108 is located in the desired position relative to extension bar 125, first upper leg support slider 134 may be secured to extension bar 125 with a fastener (not shown) such as a set screw that extends through first upper leg slider 134 and selectively engages extension bar 125.

Second upper leg support 110 may be similar or identical to first upper leg support 108. For instance, second upper leg support 110 includes a second upper leg cradle 138, cushioning 140, and a fastener 142. Additionally, the height of second upper leg support 110 may be selectively adjustable. For example, second upper leg support 110 may be connected to a second upper leg support slider 144 (FIG. 4) that is slidably positioned within track 136. A locking mechanism, such as a set screw, may selectively secure second upper leg support slider 144 in place relative to extension bar 125 and, when loosened or released, allow for the height of second upper leg support 110 to be adjusted relative to extension bar 125.

The adjustability of lower leg support 106, first upper leg support 108, second upper leg support 110, and extension bar 125 provide great versatility to mobility device 100. For instance, the positioning of each of lower leg support 106, first upper leg support 108, second upper leg support 110, and extension bar 125 may be adjusted independent of one another or in various combinations. For instance, lower leg support 106, first upper leg support 108, and second upper leg support 110 may be independently adjusted relative to extension bar 125 and one another. This allows mobility device to be adjusted for users with different femur lengths and/or desires.

Since a particular user's femur length does not typically change during the time a mobility device is used, a user may only have to adjust the relative positioning between lower leg support 106, first upper leg support 108, second upper leg support 110, and extension bar 125 once. However, the height of lower leg support 106, first upper leg support 108, and second upper leg support 110 relative to the ground may need to be periodically adjusted to account for certain variables (e.g., user height, whether shoes are being worn, height of shoes). Accordingly, the height of lower leg support 106, first upper leg support 108, and second upper leg support 110 relative to the ground may be adjusted together. Specifically, once first upper leg support 108 and second upper leg support 110 are adjusted relative to extension bar 125, and extension bar 125 is adjusted relative to lower leg support 106, the position of lower leg support 106 relative to main body 102 and/or foot 104 may be adjusted to accommodate for the above-noted variables.

Furthermore, the positions of each of lower leg support 106, first upper leg support 108, second upper leg support 110, and extension bar 125 are continuously adjustable rather than only being adjustable between discrete positions. In other words, each of lower leg support 106, first upper leg support 108, second upper leg support 110, and extension bar 125 may be slid or otherwise moved to and secured in substantially any positioned along a particularly length. As a result, mobility device 100 may be custom tailored or adjusted to any of an almost infinite number of configurations, rather than only to a few preset configurations.

In addition or as an alternative to securing second upper leg support 110 to the user's upper leg, second upper leg support 110 may be secured to the user's waist. For instance, the height of second upper leg support 110 may be sufficiently adjustable so that second upper leg support 110 may be raised to about the height of the user's waist. Additionally, first upper leg support cradle 138 may be sized and shaped to accommodate the user's hip and fastener 142 may be long enough to extend around the user's waist. To accommodate bending of the hip and/or upper leg, first upper leg support cradle 138 may be able to pivot relative to mobility device 100.

In some embodiments, a mobility device may only include a single upper leg support. For instance, either first upper leg support 108 or second upper leg support 110 may be omitted. Similarly, in other embodiments, more than two upper leg supports may be included. For instance, first upper leg support 108 and second upper leg support 110 may be included to secure the mobility device to the user's upper leg. An additional support may also be included to secure the mobility device to the user's waist. For instance, in some embodiments, a separate harness (not shown) may be attached to mobility device 100. The harness may include a waist band or belt and a connector. The waist band or belt may be secured around the user's waist and the connector (e.g., clip, strap, hook and loop fabrics) may be used to connect the harness to mobility device 100. In other embodiments, the additional support may be similar to upper leg supports 108, 110. For instance, the additional support may include a waist cradle, a waist band, or a belt that is movably connected to main body 102. For instance, the waist cradle, waist band, or belt may be connected to a telescoping component that is slidably associated with main body 102 or by a strap that connects the belt to the top or other location of the mobility device 100. At least a portion of the telescoping component may be slidably received within main body 102 such that the height of the waist cradle, waist band, or belt may be selectively adjusted to accommodate users of different heights. Securing a support (e.g., first upper leg support cradle 138 or an additional waist cradle) or a harness around the user's waist may provide for a more solid attachment of mobility device 100 to the user's body, thereby reducing the amount of play therebetween, particularly for users that are very active.

With reference to FIGS. 1, 2, 4, and 5, foot 104 will now be described. Generally, foot 104 includes a foot adapter 148 and a foot plate 150. Foot adapter 148 and foot plate 150 may be formed as discrete components and connected together in any suitable manner. For instance, foot adapter 148 and foot plate 150 may be connected together with screws, nuts and bolts, clamps, welding, adhesives, and the like. Alternatively, foot adapter 148 and foot plate 150 may be formed as a unitary component.

Foot adapter 148 is connected to main body 102, thereby linking foot plate 150 to main body 102. Foot adapter 148 may be connected to main body 102 in a variety of ways. In the illustrated embodiment, for instance, a portion of foot adapter 148 extends into channel 120 through an opening in the lower end of main body 102 and is secured in place. In other embodiments, foot adapter 148 may be bolted, screwed, clamped, welded, glued, or otherwise secured to main body 102. Additionally, foot adapter 148 may not extend into channel 120, but rather may be connected to a side or end of main body 102. Still further, foot adapter 148 may be adjustably connected to main body 102 to enable the height of main body 102 to be adjusted relative to foot plate 150. For instance, foot adapter 148 may be slidably received within channel 120 and a locking mechanism may be used to selectively secure foot adapter 148 in various positions within channel 120.

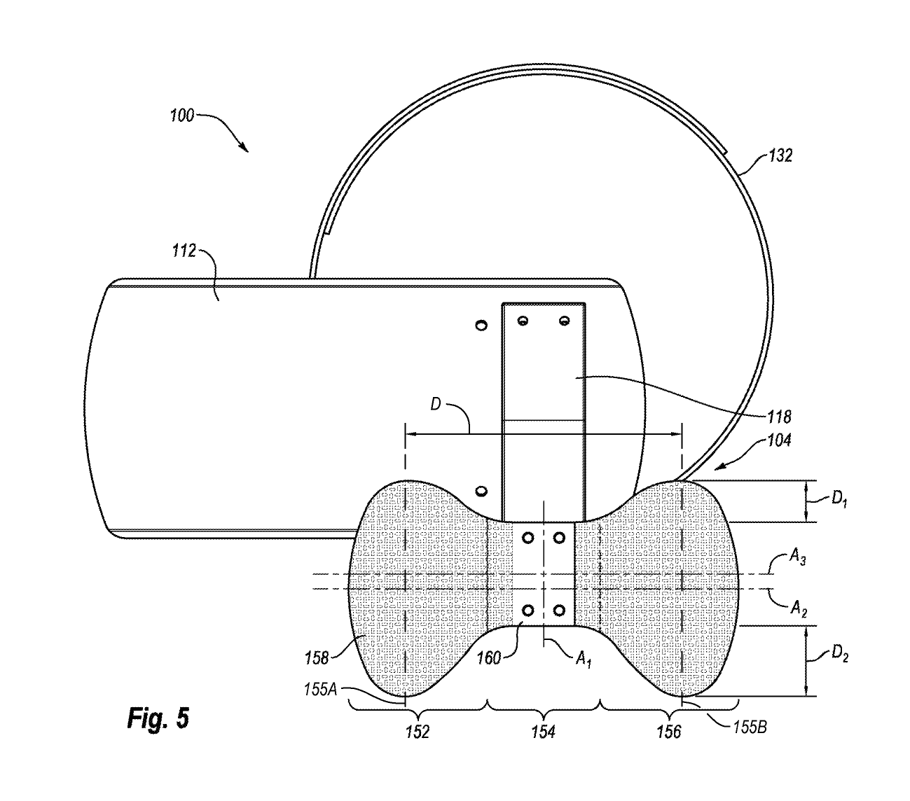

In the illustrated embodiment, foot 104 includes the three general regions identified in FIGS. 2 and 5. The three general regions include a rear region 152, a middle region 154, and a forward region 156. As best seen in FIG. 2, rear region 152 is generally horizontally offset rearwardly from main body 102. For instance, rear region 152 may be substantially or entirely positioned rearward of a vertical projection of main body 102. Middle region 154 is generally horizontally aligned with and positioned vertically below main body 102. For instance, middle region 154 may be substantially or entirely positioned within or aligned with a vertical projection of main body 102. Like rear region 152, forward region 156 is horizontally offset from main body 102. However, forward region 156 is generally horizontally offset from main body 102 in a forward direction. For instance, forward region 156 may be substantially or entirely positioned forward of a vertical projection of main body 102.

Accordingly, foot 104 comprises a split foot arrangement. That is, a first portion (i.e., rear region 152) extends rearwardly of main body 102 from middle region 154 and a second portion (i.e., forward region 156) extends forwardly of main body 102 from middle region 154. In the illustrated embodiment, middle region 154 of foot plate 150 is connected via foot adapter 148 to main body 102 such that middle region 154 generally bisects foot plate 150. As a result of the illustrated split foot arrangement, foot 104 has two lines of contact 155A, 155B with the ground. That is, rear region 152 contacts the ground at a first line of contact 155A and forward region 156 contacts the ground at a second line of contact 155B. Having multiple lines of contact between foot 104 and the ground may provide greater stability and may more closely simulate a user's real foot compared to a relatively small, single line or point of contact.

As illustrated in FIG. 5, lines of contact 155A, 155B are spaced apart from one another a distance D. In some exemplary embodiments, distance D is between about 3 inches and about 5.5 inches, between about 3.5 inches and about 5 inches, and between about 4 inches and about 4.5 inches. In one embodiment, distance D is about 4.25 inches. In another embodiment, distance D is about 4.375 inches.

The spacing (i.e., distance D) between lines of contact 155A, 155B can affect the performance of foot 104. For instance, by spacing lines of contact 155A, 155B sufficiently far enough apart, foot 104 can provide a stable platform for mobility device 100. That is, when a user is standing with mobility device 100, the spaced arrangement of lines of contact 155A, 155B allows the user to stand without requiring undue effort on the user's part to maintain his or her balance. Whereas, if there were a single point of contact or the lines of contact were too close together, the foot may not provide a stable base. Nevertheless, by spacing lines of contact 155A, 155B close enough together enables foot 104 to roll smoothly as a user walks with mobility device 100. If lines of contact 155A, 155B are too far apart, then the user will experience an unnatural thud as the user transitions from a heel strike (e.g., with rear region 152) to forward region 156 (e.g., as foot 104 rolls forward so that both lines of contact 155A, 155B are in contact with the ground). Similarly, if lines of contact 155A, 155B are too far apart, extra effort is required to roll foot 104 forward when pushing off the ground.

In the illustrated embodiment, lines of contact 155A, 155B are generally parallel to one another. Additionally, lines of contact 155A, 155B extend between opposing sides of foot 104 and are oriented generally perpendicularly to the length of foot 104. It is understood, however, that the illustrated configuration is merely exemplary. In other embodiments, for instance, lines of contact 155A, 155B may not be parallel to one another. Likewise, one or both of lines of contact 155A, 155B may not be oriented generally perpendicularly with the length of foot 104. Still further, while lines of contact 155A, 155B are illustrated as being about the same length as one another, lines of contact 155A, 155B may have difference lengths from one another. For instance, line of contact 155A may be longer or shorter than line of contact 155B, and vice versa.

As noted above, foot adapter 148 and foot plate 150 may be formed from one or more components. Similarly, foot plate 150 may be formed as a unitary component or from multiple discrete components that are joined together. For instance, rear region 152, middle region 154, and forward region 156 may be integrally formed or may be individually formed and joined together. Regardless of whether a unitary component or multiple discrete components are used to form foot 104 and/or foot plate 150, foot 104 may still have a split foot configuration. That is, the split foot configuration of foot 104 includes a portion of foot 104 (e.g., rear region 152) that extends rearward of main body 102 and a portion of foot 104 (i.e., forward region 156) that extends forward of main body 102, regardless of whether those portions are part of a unitary component or separate components.

In some embodiments, rear region 152 extends rearward of main body 102 a distance that is generally equal to or greater than a distance between rearward and forward ends of main body 102, foot adapter 148, and/or middle region 154. Similarly, in some embodiments, forward region 156 extends forward of main body 102 a distance that is generally equal to or greater than a distance between rearward and forward ends of main body 102, foot adapter 148, and/or middle region 154. Furthermore, rear region 152 may extend rearward of main body 102 a first distance and forward region 156 may extend forward of main body 102 a second distance. In some embodiments, the first distance is generally equal to the second distance. In other embodiments, the first distance and the second distance are different from one another. For instance, the first distance may be greater or less than the second distance and vice versa.

As can be seen best in the FIGS. 1 and 2, middle region 154 is generally planar, but may be otherwise shaped or configured. Additionally, when foot 104 is positioned on a support surface (e.g., floor, ground), middle region 154 may be spaced apart from the support surface, while at least a portion of rear region 152 and forward region 156 contact the support surface. The spacing of middle region 154 apart from the support surface is, at least in part, attributable to the shape or configuration of rear region 152 and forward region 156.

Rear region 152 and forward region 156 may each have a generally arcuately shaped profile, as seen in FIG. 2. The arcuately shaped profiles of rear region 152 and forward region 156 allow for foot 104 to more realistically mimic the user's real foot, thereby providing a smoother and more natural walking experience for the user. This curvature may provide shock absorption and flexibility to the foot 104.

More specifically, rear region 152 extends downwardly and rearwardly from middle region 152 to line of contact 155A and then upwardly and rearwardly from line of contact 155A. The portion of rear region 152 that extends upwardly and rearwardly from line of contact 155A may act as a heel and/or ankle (referred to hereinafter as the “heel” of foot 104). By way of example, as the user walks and plants foot 104 on the ground, the heel or arcuately shaped profile of rear region 152 allows mobility device 100 to smoothly pivot or roll forward, thereby mimicking the actions normally provided by the user's heel and ankle.

Similarly, forward region 156 extends downwardly and forwardly from middle region 152 to line of contact 155B and then upwardly and forwardly from line of contact 155B. The portion of forward region 156 that extends upwardly and forwardly may simulate the bending of the user's natural foot and toes (referred to hereinafter as the “toe portion” of foot 104). By way of example, as the user walks and pushes off of the ground, the toe portion or arcuately shaped profile of forward region 156 allows mobility device 100 to smoothly pivot or roll forward, thereby mimicking the flexing and bending of the user's natural foot and toes. Thus, the combination of the rearward and forward regions 154, 156 may act as a replacement for the user's ankle, heel, and toes.

In some embodiments, the curvature or arcuate shape of all or a portion of the heel and toe portions of foot 104 may define or lie within an arc. Furthermore, in some embodiments, lines of contact 155A, 155B may be tangent to the arc formed by the heel and toe portions of foot 104.

The profile and the material used to form foot 104, and particularly foot plate 150, may also provide some shock absorbing characteristics to mobility device 100. For instance, foot plate 150 may be formed of a material, such as a carbon fiber, that is at least somewhat flexible. Forming foot plate 150 of an at least slightly flexible material may allow rear region 152 and forward region 156 to at least slightly deflect relative to middle region 154. As a result, when user walks or shifts his weight onto foot 104, rear region 152 and/or front region 156 may slightly deflect relative to middle region 154 to absorb at least some of the forces associated with the movement. Notably, having middle region 154 spaced apart from the ground allows middle region 154 to move vertically lower when rear region 152 and forward region 156 deflect. Enabling middle region 154 to move vertically before middle region contacts the ground provides some of the shock absorbing characteristics of foot 104.

It is understood, however, that in some embodiments middle region 154 may not be spaced apart from the ground. In such embodiments, rear region 152 may extend rearwardly, but not downwardly, from middle region 152 before extending upwardly and rearwardly from the portion of rear region 154 that contacts the support surface. Similarly, forward region 156 may extend forwardly, but not downwardly, from middle region 152 before upwardly and forwardly from the portion of forward region 156 that contacts the support surface. As a result, foot plate 150 may have a generally flat profile except for the portion of rear region 152 that extends upwardly and rearwardly (i.e., the portion that acts as a heel and/or ankle) and the portion of forward region 156 that extends upwardly and forwardly (i.e., the portion that acts as toes).

As shown in FIG. 5, foot 104 may have a footprint that is generally symmetrical across axis A₁. As also shown in FIG. 5, middle region 154 may be narrower from side to side than rear region 152 and front region 156. The widths of rear region 152 and front region 156 may taper closer together near middle region 154. In other embodiments, middle region 154 may be as wide or wider from side to side as rear region 152 and/or front region 156.

One or both of rear region 152 and front region 156 may extend laterally further relative to a first side of middle region 154 than relative to a second side of middle region 154. More specifically, one or both of rear region 152 and front region 156 may extend medially in a first direction a distance D₁ relative to a first side of middle region 154. Similarly, one or both of rear region 152 and front region 156 may extend laterally in a second direction a distance D₂ relative to a second side of middle region 154. As can be seen in FIG. 5, distance D₂ is greater than distance D₁. In some embodiments, one or both of rear region 152 and front region 156 may not extend medially in the first direction relative to the first side of middle region 154. In other words, D₁ may be equal to zero (0).

In other words, foot 104 may have a central longitudinal axis A₂ that extends between rear and front ends of foot 104, and middle region 154 may have a central axis A₃ that is generally parallel to and horizontally offset from axis A₂. As seen in FIG. 4, central axis A₃ of middle region 154 lies in generally the same plane as a central longitudinal axis A₄ of main body 102. As a result, central axis A₂ of foot 104 is laterally offset from both central axis A₃ of middle region 154 and central longitudinal axis A₄ of main body 102.

When mobility device 100 is secured to a user's leg as shown in FIG. 1, main body 102 is positioned along a lateral side of the user's upper leg. That is, central longitudinal axis A₄ of main body 102 is generally parallel to, but laterally offset from a central axis (not shown) of the user's upper leg. As a result, when the user's weight is applied to lower leg support 106, mobility device 100 will tend to tilt medially towards the user. This tilting of mobility device 100 will cause first upper leg support 108 and second upper leg support 110 to push the user towards their center of gravity, which will increase the user's stability. By laterally offsetting axis A₂ of foot 104 even further away from the user's upper leg than longitudinal axis A₄ of main body 102, the stabilizing effect may be further enhanced.

In addition to the foregoing, foot 104 may also include tread 158 and a support bracket 160. Tread 158 may comprise a slip-resistant material or surface texturing applied to the bottom surface of foot plate 150. For instance, tread 158 may comprise textured rubber secured to the bottom surface of foot plate 150.

Foot 104 may also include a support bracket 160. Support bracket 160 may be positioned under foot plate 150 and/or tread 158 near middle region 154. Support bracket may facilitate connection of foot plate 150 to foot adapter 148. For instance, fasteners, such as bolts, may extend through support plate 160 and foot plate 150 and into or through foot adapter 148.

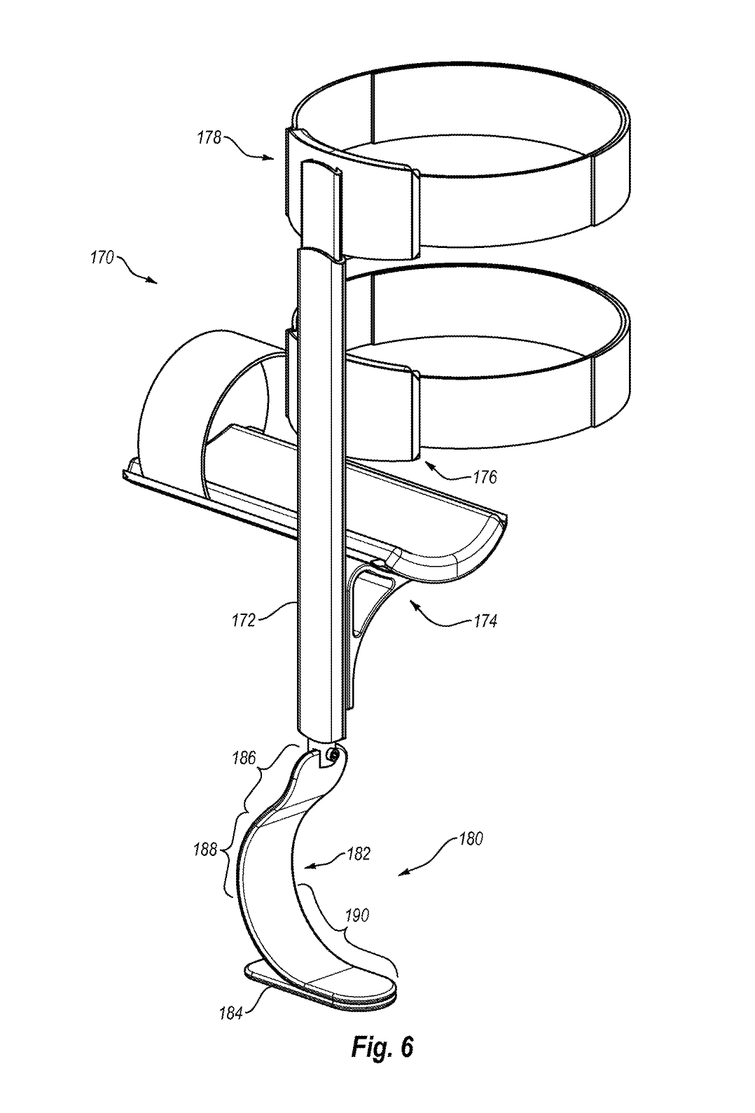

Attention is now directed to FIG. 6, which illustrates a mobility device 170 according to another exemplary embodiment of the present invention. Many aspects of mobility device 170 are similar or identical to mobility device 100. For instance, mobility device 100 includes a main body 172, a lower leg support 174, a first upper leg support 176, and a second upper leg support 178 that are similar or identical to main body 102, lower leg support 106, first upper leg support 108, and second upper leg support 110.

Mobility device 170 also includes a foot 180. Foot 180 is generally a two part foot. More specifically, foot 180 includes a first foot portion 182 and a second foot portion 184. Similar to foot 104, first foot portion 182 is connected to a lower end of main body 172. Second foot portion 184 is connected to a lower end of first foot portion 182 and is configured to engage the ground or other support surface. In some embodiments, second foot portion 184 is omitted.

First foot portion 182 includes multiple curved regions. For instance, first foot portion 182 includes a first curved region 186 near the upper end thereof. First curved region 186 extends downwardly and rearwardly from the lower end of main body 172. The curve of first curved region 186 opens upwardly (i.e. rearward and upward). A second curved region 188 extends from the lower end of first curved region 186. Second curved region 188 extends downwardly and rearwardly from the lower end of first curved region 186. The curve of second curved region 188 opens downwardly (i.e. forward and downward). A third curved region 190 extends from the lower end of second curved region 188. Third curved region 190 extends downwardly and forwardly from the lower end of second curved region 188. The curve of third curved region 190 opens upwardly (i.e. forward and upward). Second curved region 188 and third curved region 190 together have a generally C-shaped profile. As can be seen, at least a portion of each of first curved region 186, second curved region 188, and third curved region 190 are positioned rearward of main body 172, while at least a portion of the lower end of third curved region is positioned forward of main body 172.

As noted above, second foot portion 184 is connected to a lower end of first foot portion 182 and is configured to engage the ground or other support surface. More specifically, a forward end of second foot portion 184 is connected to the lower and forward end of first foot portion 182 and extends rearwardly therefrom. As a result, a portion of second foot portion 184 is positioned forward of main body 172 and a portion is positioned rearward of main body 172. Second foot portion 184 may be generally planar or may have one or more curves formed therein. Additionally, second foot portion 184 may have a slip-resistant tread or surface texturing on a bottom surface thereof.

As a user stands or walks on foot 180, first foot portion 182 may flex or deflect to absorb at least some of the forces associated with the user's movements. When first foot portion 182 flexes or deflects, the generally C-shaped profile of second curved region 188 and third curved region 190 may compress or deform. Second foot portion 184 may also flex or deflect to absorb some of the forces associated with the user's movements. For instance, as the user walks and plants foot 180 on the ground, the rearward end of second foot portion 184 may flex or deflect to absorb at least some of the impact forces associated with planting foot 180 on the ground.

In the illustrated embodiment, foot 180 is generally laterally aligned with main body 172. It will be appreciated, however, that this is merely exemplary. Foot 180 may be medially and/or laterally offset from main body 172 in order to obtain the enhanced stabilizing affects discussed above in connection with mobility device 100.

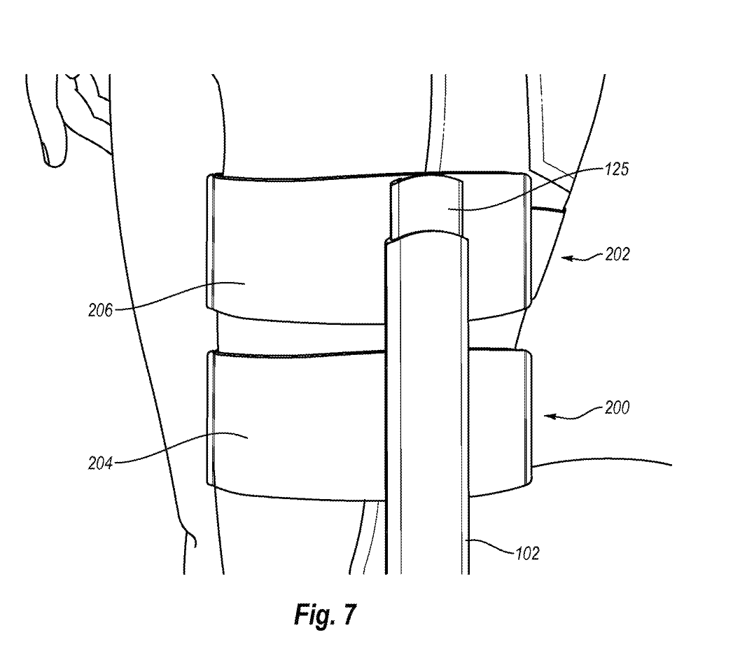

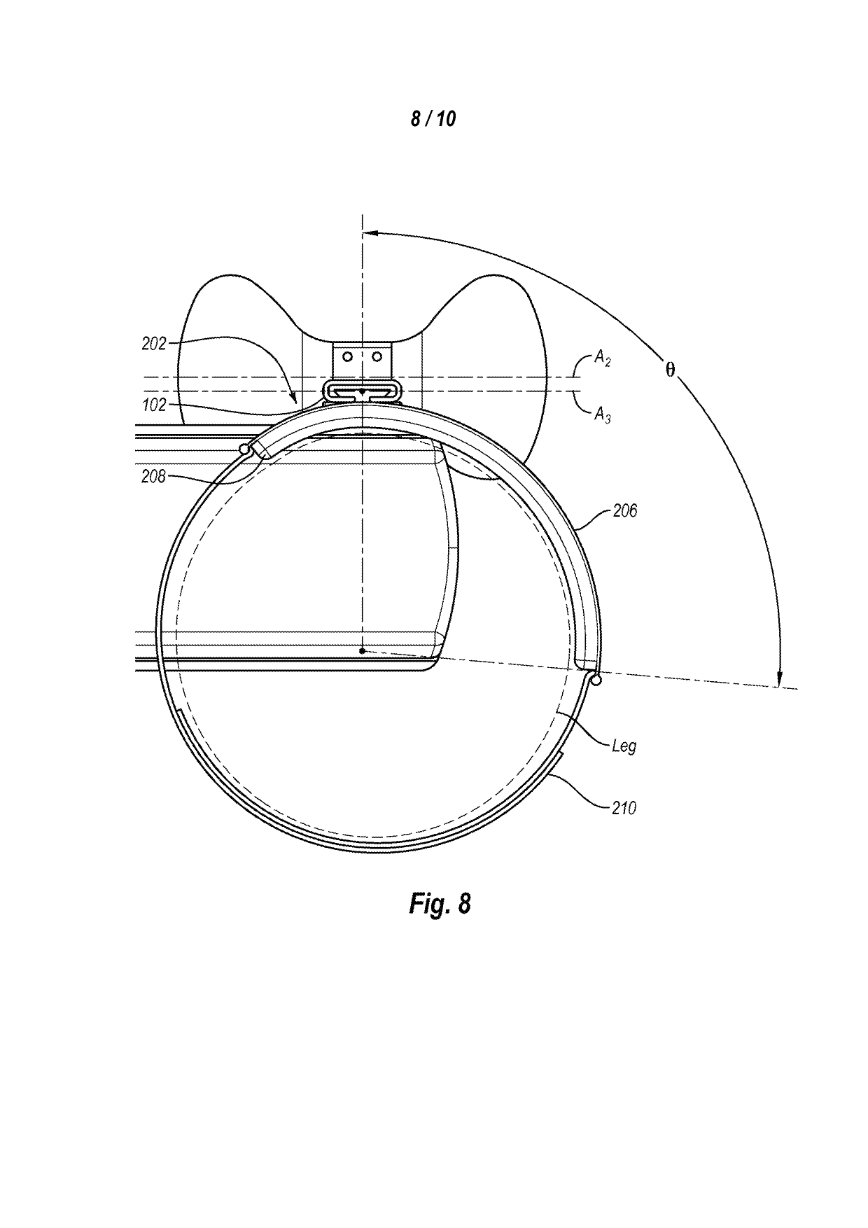

With attention to FIGS. 7 and 8, alternative embodiments of upper leg supports will now be described. While the upper leg supports illustrated in FIGS. 7 and 8 may be used in connection with any of the mobility devices disclosed herein, the upper leg supports are illustrated in connection with mobility device 100. That is, the upper leg supports illustrated in FIGS. 7 and 8 have replaced upper leg supports 108, 110.

FIGS. 7 and 8 illustrate a first upper leg support 200 and a second upper leg support 202. First upper leg support 200 and second upper leg support 202 are designed to secure a mobility device to the user's upper leg and/or waist. First upper leg support 200 includes a generally rigid first upper leg cradle 204 that is designed to be positioned against and extend at least partially around the user's upper leg. First upper leg support 200 may include cushioning for comfort and a fastener to secure the user's upper leg to first upper leg support 200. Similarly, second upper leg support 202 includes a generally rigid second upper leg cradle 206 that is designed to be positioned against and extend at least partially around the user's upper leg. Second upper leg support 202 also includes cushioning 208 for comfort and a fastener 210 to secure the user's upper leg to second upper leg support 202.

Different from upper leg support cradles 128, 138, upper leg support cradles 204, 206 extend further around the front of the user's upper leg. For instance, FIG. 8 illustrates a top view of upper leg support 202 attached to a user's leg. As can be seen, upper leg support cradle 206 extends at least partially around and toward the front of the user's leg. For instance, upper leg support cradle 206 may extend through an angle θ between an attachment point (i.e., where upper leg support cradle 206 connects to main body 102) and a location along the front of the user's leg. The angle θ may be less than 90°, generally equal to 90°, or greater than 90°. In the illustrated embodiment, for instance, the angle θ is between about 90° and about 100°.

As a result of this configuration of upper leg support cradles 204, 206, when the user swings his leg forward, the leg pushes against upper leg support cradles 204, 206. Since upper leg support cradles 204, 206 are generally rigid and securely connected to the rest of the mobility device, the forces from the user's leg are better transferred to the mobility device. As a result, the mobility device may respond to the movements of the user's leg more quickly and smoothly, with less relative movement between the user's leg and the mobility device.

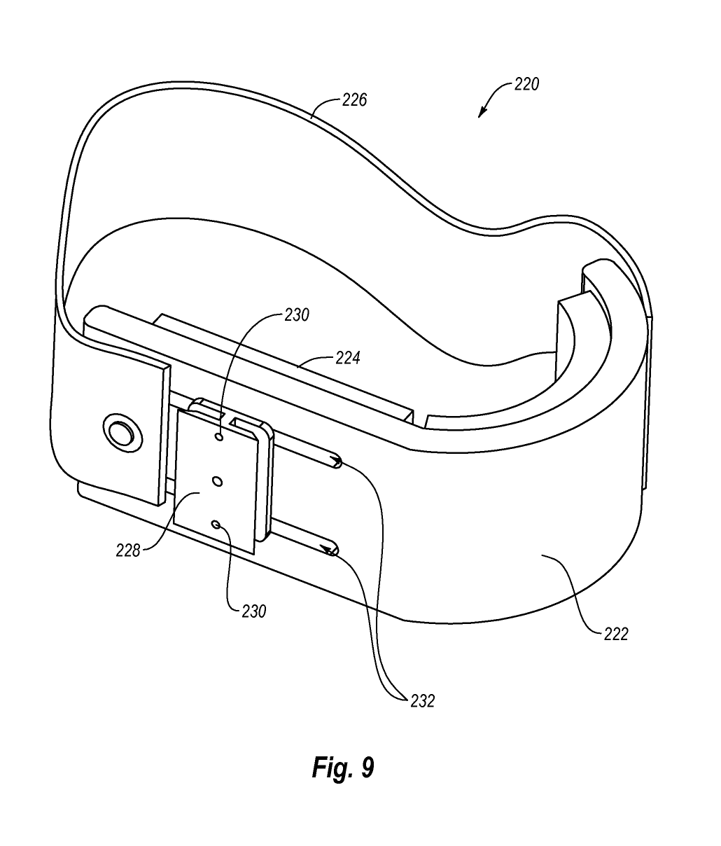

With attention to FIG. 9, an alternative embodiment of an upper leg support will now be described. The upper leg support illustrated in FIG. 9 may be used in connection with any of the mobility devices disclosed herein. That is, the upper leg support illustrated in FIG. 9 may be used in connection with mobility devices 100, 170, such as an alternative to upper leg supports 108, 110, 176, 178, 200, 202.

FIG. 9 illustrates an upper leg support 220 that is designed to secure a mobility device to the user's upper leg and/or waist. Upper leg support 220 includes a generally rigid cradle 222 that is designed to be positioned against and extend at least partially around the user's upper leg and/or waist. In the illustrated embodiment, upper leg support cradle 222 is designed to extend around the front of the user's upper leg in much the same way as upper leg support cradles 204, 206. Nevertheless, upper leg support cradle 222 may be shaped and configured similar to upper leg support cradles 128, 138. Upper leg support 220 may include cushioning 224 for comfort and a fastener 226 to secure the upper leg support 220 to the user's upper leg or waist.

When connected to a mobility device, such as mobility devices 100, 170, the height of upper leg support 220 may be selectively adjustable. Similar to the other upper leg supports described herein, upper leg support 220 may be connected to an upper leg support slider 228 that may be slidably positioned within track 136 (see FIG. 4). A locking mechanism (e.g., screws, nuts and bolts, quick release bolts, clamps, etc.) may selectively secure upper leg support slider 228 in place relative to extension bar 125 and, when loosened or released, allow for the height of upper leg support 220 to be adjusted relative to extension bar 125.

In addition to the height of upper leg support 220 being adjustable along the length of extension bar 125 and/or main body 102, the fore and aft position of upper leg support 220 is also selectively adjustable. More specifically, the location on upper leg support cradle 222 where upper leg support slider 228 is connected may be adjusted. When the relative positions of upper leg support cradle 222 and upper leg support slider 228 are adjusted, the position of upper leg support cradle 222 will be adjusted relative to main body 102 and extension bar 125. Such adjustments can be made to accommodate different users. For instance, a user with a larger leg may need upper leg support cradle 222 to be positioned further forward relative to main body 102 so that main body 102 is positioned along the side of the user's upper leg. In contrast, a user with a smaller leg may need upper leg support cradle 222 to be positioned further back relative to main body 102 so that main body 102 is positioned along the side of the user's upper leg.

FIG. 9 illustrates one example manner for allowing the fore and aft adjustment of upper leg support 220 relative to main body 102. In the illustrated embodiment, upper leg support slider 228 is connected to upper leg support cradle 222 with two fasteners 230 (e.g., screws, nuts and bolts, quick release bolts, etc.). Fasteners 230 extend through two longitudinal slots 232 in upper leg support cradle 222. When fasteners 230 are tightened, upper leg support slider 228 is secured in place on upper leg support cradle 222. When fasteners 230 are loosened, the relative fore and aft position of upper leg support cradle 222 may be adjusted relative to upper leg support slider 228. Fasteners 230 may slide through slots 232 while the relative fore and aft positioning of upper leg support slider 228 and upper leg support cradle 222 is adjusted. Once upper leg support slider 228 and upper leg support cradle 222 are positioned as desired, fasteners 228 may be tightened to secure upper leg support slider 228 and upper leg support cradle 222 relative to one another.

It will be understood by one of skill in the art that the fore and aft adjustment of upper leg support 220 may be accomplished with mechanisms other than slots 232. By way of example, upper leg support cradle 222 may include a series of spaced holes through which fasteners 230 may extend to connect upper leg support cradle 222 to upper leg support slider 228. The hole(s) through which fasteners 230 extend can determine the position of upper leg support cradle 222 relative to upper leg support slider 228.

In addition or as an alternative to actually changing the position of upper leg support cradle 222 relative to upper leg support slider 228, more or less cushioning 224 can be used inside the front (e.g., the arcuate portion) of upper leg support cradle 222. Adding or removing cushioning 224 will change the position of the user's upper leg relative to main body 102, thereby allowing the user to customize the mobility device to his or her desire and needs.

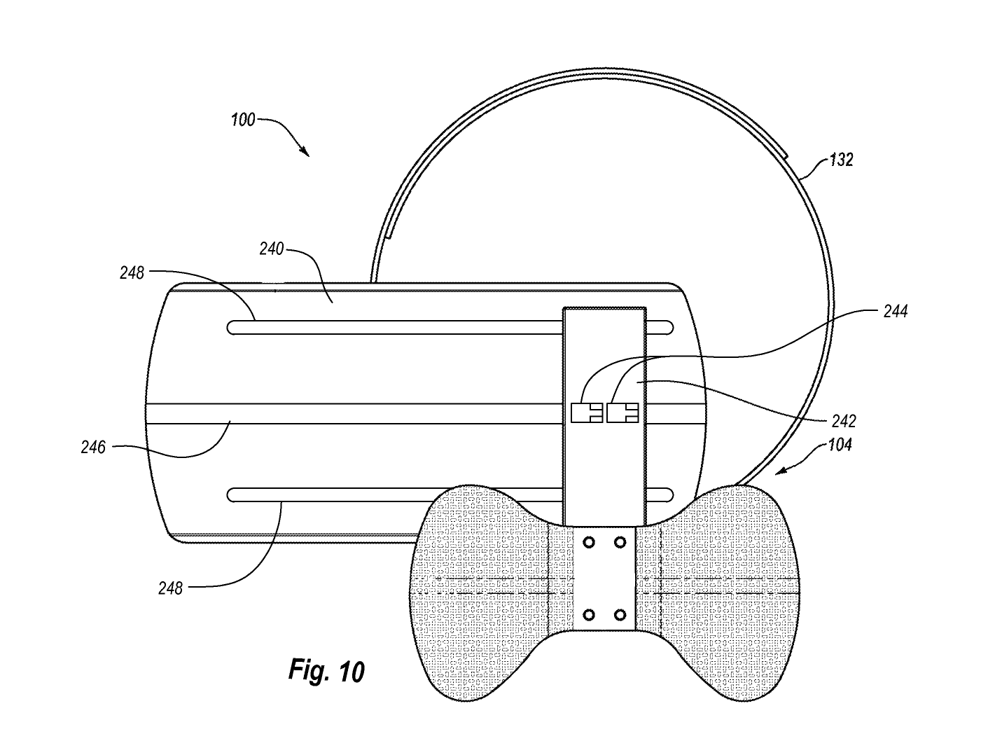

Similar to the adjustable nature of upper leg support 220, a lower leg support may also be adjustable in the fore and aft directions relative to the main body of a mobility device. FIG. 10 illustrates one example of an adjustable lower leg support 240 and associated platform 242 that may be used in connection with any of the mobility devices described herein. Accordingly, lower leg support 240 and platform 242 can be similar to lower leg support 112 and platform 118 or lower leg support 174 in many respects.

Unlike lower leg support 112 and platform 118, which are bolted or screwed together, lower leg support 240 and platform 242 are secured together such that lower leg support 240 may be slidably adjusted fore and aft relative to platform 242. Such adjustability can enable a mobility device to be further customized to a particular user's needs and preferences. Additionally, such adjustability allows a mobility device to be quickly and easily transformed for use on either a right leg or a left leg. Additionally, the adjustable nature of lower leg support 112 may allow the mobility device to be arranged in a more compact configuration, such as for shipping and storage. For instance, lower leg support 112 can be readily adjusted relative to or detached from main body 102 so that the mobility device can be packaged in a smaller container or stored in a smaller space.

FIG. 10 illustrates one example manner for allowing the fore and aft adjustment of lower leg support 240 relative to platform 242. In the illustrated embodiment, lower leg support 240 is connected to platform 242 with two fasteners 244 (e.g., screws, nuts and bolts, quick release bolts, etc.). In other embodiments, one or more than two fasteners may be used to connect lower leg support 240 to platform 242. Fasteners 244 extends through platform 242 and into a lower leg support slider (not shown, but which may be similar to the other sliders shown and described herein). The lower leg support slider can nest or ride at least partially within a slot 246 formed in the underside of lower leg support 240, thereby connecting together lower leg support 240 and platform 242. When fasteners 244 are tightened, lower leg support 240 is secured in place on platform 242. When fasteners 244 are loosened, the fore and aft positioning of lower leg support 240 relative to platform 242 may be adjusted. The lower leg support slider may slide through slot 246 while the fore and aft position of lower leg support 240 is adjusted relative to platform 242. Once lower leg support 240 is positioned as desired relative to platform 242, fastener 244 may be tightened to secure lower leg support 240 in place relative to platform 242.

Thus, a user may selectively adjust the position of lower leg support 240 so that the mobility device is comfortable and suits the particular user. Additionally, slot 246 may be long enough that lower leg support 240 can be adjusted for use with either a right leg or a left leg. In some embodiments, including the embodiment illustrated in FIG. 10, slot 246 may extend the entire length of lower leg support 240, such that the lower leg support slider can be inserted or removed from one or both ends of slot 246.

As illustrated, lower leg support 240 is secured on platform 242 so that mobility device 100 can be used on a person's left leg. However, lower leg support 240 can be adjusted relative to platform 242 (e.g., so that fastener 244 is positioned within an opposing end of slot 246) so that mobility device 100 can be used on a person's right leg.

The movement of lower leg support 240 may be constrained to fore and aft movement relative to platform 242. As will be understood by one of skill in the art, using multiple aligned fasteners 244 to connected lower leg support 240 to platform 242 (as shown in FIG. 10) can limit the movement of lower leg support 240 to movement within a single line.

In addition or as an alternative to using multiple fasteners to constrain the movement of lower leg support 240, lower leg support 240 may also include one or more additional slots 248 formed in a bottom surface thereof. Slots 248 may receive therein one or more guide pins (not shown) that extend upwardly from platform 242. The guide pins and slots 248 may cooperate to maintain proper alignment of lower leg support 240 with platform 242, particularly when lower leg support 240 is adjusted relative to platform 242. Additionally or alternatively, the curved interfacing surfaces of lower leg support 240 (e.g., the curved bottom surface of lower leg support 240) and platform 242 (e.g., the curved top surface of platform 242) can cooperate to maintain proper alignment between lower leg support 240 and platform 242.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

What is claimed is:

1. A mobility device, comprising:
a main body having a lower end, an upper end, and a longitudinal axis extending therebetween, said main body being selectively securable to a lateral side of a user's leg;
a lower leg support connected to said main body, said lower leg support being configured to have a portion of the user's lower leg positioned thereon and selectively secured thereto, said lower leg support being movably connected to said main body such that a fore and aft position of said lower leg support relative to said main body is selectively adjustable;
one or more upper leg supports connected to said main body, said one or more upper leg supports being configured to selectively secure said upper end of the main body to a portion of the user's upper leg; and
a foot connected to said lower end of said main body.

2. The mobility device of claim 1, wherein said lower leg support is movably connected to said main body such that a distance between said lower leg support and said foot is selectively adjustable.

3. The mobility device of claim 1, wherein said lower leg support is adjustable between a first configuration and a second configuration, wherein said mobility device is configured for use with a user's left leg when said lower leg support is in the first configuration, and wherein said mobility device is configured for use with a user's right leg when said lower leg support is in the second configuration.

4. The mobility device of claim 1, wherein said lower leg support is slidably connected to a platform that is connected to said main body.

5. The mobility device of claim 1, wherein said foot has a split foot configuration with a first portion of said foot being positioned rearward of said main body and a second portion of said foot being positioned forward of said main body.

6. The mobility device of claim 5, wherein said first portion of said foot defines a first line of contact with a support surface and said second portion of said foot defines a second line of contact with the support surface.

7. The mobility device of claim 6, wherein said first line of contact and said second line of contact are spaced apart from one another by a distance of between about 3 inches and about 5.5 inches.

8. The mobility device of claim 1, wherein at least one of said one or more upper leg supports is movably connected relative to said main body such that a position of said at least one upper leg support is selectively adjustable fore and aft relative to said main body.

9. The mobility device of claim 8, wherein said at least one upper leg support comprises a slot that facilitates the selectively fore and aft adjustment of said at least one upper leg support.

10. A mobility device, comprising:
a main body having a lower end, an upper end, and a longitudinal axis extending therebetween, said main body being selectively securable to a lateral side of a user's leg;
a lower leg support connected to said main body, said lower leg support being configured to have a portion of the user's lower leg positioned thereon and selectively secured thereto;
an upper leg support connected to said main body, said upper leg support being configured to selectively secure said upper end of the main body to a portion of the user's upper leg, said upper leg support being movably connected relative to said main body such that a fore and aft position of said upper leg support relative to said main body is selectively adjustable; and
a foot connected to said lower end of said main body.

11. The mobility device of claim 10, wherein said upper leg support is linked to said main body by an upper leg support slider.

12. The mobility device of claim 11, wherein said upper leg support is slidably connected to said upper leg support slider.

13. The mobility device of claim 10, wherein said upper leg support comprises an upper leg support cradle having one or more slots formed therein to facilitate the selective adjustment of the fore and aft position of said upper leg support.

14. The mobility device of claim 10, wherein said foot defines a first line of contact with a support surface and a second line of contact with the support surface.

15. The mobility device of claim 14, wherein said first line of contact and said second line of contact are spaced apart from one another by a distance of between about 4 inches and about 4.5 inches.

16. The mobility device of claim 10, wherein said lower leg support is movable relative to said main body such that a fore and aft position of said lower leg support is selectively adjustable relative to said main body.

17. The mobility device of claim 10, wherein said lower leg support is adjustable relative to said main body between a first configuration and a second configuration, wherein said mobility device is configured for use with a user's left leg when said lower leg support is in the first configuration, and wherein said mobility device is configured for use with a user's right leg when said lower leg support is in the second configuration.

18. A mobility device for providing mobility to a user with a lower leg in a non-functioning condition, comprising:
a main body having a lower end, an upper end, and a longitudinal axis extending therebetween, said main body being selectively securable to a lateral side of the user's non-functioning leg;
a lower leg support connected to said main body, said lower leg support being configured to have a portion of the user's non-functioning lower leg positioned thereon and selectively secured thereto;
an upper leg support connected to said main body, said upper leg support being configured to selectively secure said upper end of said main body to a portion of the user's upper leg; and
a foot connected to said lower end of said main body, said foot comprising a first foot portion and a second foot portion, said first foot portion defining a first line of contact with a support surface, said second foot portion defining a second line of contact with the support surface, said first line of contact and said second line of contact being spaced apart from one another.

19. The mobility device of claim 18, wherein said first line of contact and said second line of contact are spaced apart from one another by a distance of about 4.25 inches.

20. The mobility device of claim 18, wherein said first line of contact and said second line of contact are generally parallel to one another.
